(19) United States
(12) Patent Application Publication
(10) Pub. No.: US 2007/0071705 A1
(43) Pub. Date: Mar. 29, 2007

(54) Topical anti-microbial compositions

(76) Inventors: Monica Alves Mariani De Oliveira, Aruja (BR); Paula Giorgi, Sao Paulo (BR); Kathleen L. Clark, Medusa, NY (US); Jeffery S. Reynolds, Greenville, NY (US)

Correspondence Address: NATH & ASSOCIATES, 112 South West Street, Alexandria, VA, 22314, US

(21) Appl. No.: 11/238,021

(22) Filed: Sep. 29, 2005

(51) Int. Cl. A61K 31/44; A61K 8/73

(52) U.S. Cl. 424/70.13; 424/401; 514/345

(57) ABSTRACT

Topical anti-microbial compositions, and more particularly anti-bacterial and anti-fungal compositions, comprising a therapeutically effective amount of an anti-microbial agent, a starch, a hydroalcoholic solvent, and a volatile silicone compound having a viscosity of about 0.1 to about 50 centistokes. These compositions are capable of temporarily or permanently reducing, inhibiting, treating, ameliorating, or preventing microbial skin infections, as well as other related skin conditions. These compositions are further capable of restoring or repairing a skin lipid barrier of a mammal.

### FIELD OF THE INVENTION

The present subject matter relates generally to topical anti-microbial compositions. In a preferred embodiment, the present anti-microbial compositions comprise a therapeutically effective amount of an anti-microbial agent, a starch, a hydroalcoholic solvent, and a volatile silicone compound having a viscosity of about 0.1 to about 50 centistokes. These compositions are capable of temporarily or permanently reducing, inhibiting, treating, ameliorating, or preventing a skin infection caused by microbes, such as fungi and bacteria. The present compositions are further capable of reducing, inhibiting, treating, ameliorating, or preventing discomfort, itching, and inflammation associated with microbial infections.

### BACKGROUND OF THE INVENTION

Microbial infections are not only irritating, itchy, and painful, but they also can be socially embarrassing. Certain microbial infections are caused by dermatophytes (i.e. fungi), which infect an area of skin on a mammal and can cause inflammation, discoloration, and/or local swelling of the infected and surrounding areas. Dermatophyte infections (i.e. fungal infections) can have a variety of different causes, including poor hygiene and unsanitary living conditions.

However, dermatophyte infections can also be a side effect of physiological changes within the mammal. For example, the cause of seborrheic dermatitis, which is a disease that causes inflammation and flaking of the skin, is not known. However, two possible theories hypothesize that the disease can be caused directly by an increased exposure to malassezia (a fungi normally present on the skin of a mammal in small amounts), resulting in higher amounts of the fungi being present on the skin, or indirectly by a hormonal change in the mammal resulting in an increased amount of malassezia present on the skin. In either scenario, the end result is an increased amount of the fungi malassezia on the skin of the mammal, which leads to seborrheic dermatitis.

Another type of dermatophyte infection is onychomycosis (i.e. tinea unguium), a disease that affects the nail beds of fingernails and toenails, and causes discoloration and irregular growth of the affected nails. The disease, which can be caused by, e.g., T. rubrum, T. mentagrophytes, Candida albicans and/or Candida parapsilosis, usually begins at the corner of the nail as a yellowish discoloration. As it spreads, the nail typically begins to separate from its bed distally, resulting in irregular nail dystrophy. Healthy individuals can contract such a condition through exposure to the afflicting fungi, which is at times present in unsanitary environments. However, individuals with compromised immune systems are at a higher risk at contracting the disease since these individuals lack the ability to fight off the fungi.

Additional irritating dermatophyte infections include ringworm of the body (i.e. tinea corporis), ringworm of the foot (i.e. tinea pedis or athletes foot), ringworm of the groin (i.e. tinea cruris), sun fungus (i.e. tinea versicolor and/or pityriasis versicolor), and other external fungal infections (i.e. Candida/Monilia infections). All of these conditions can be caused by microbial fungi, including fungi of the Tinea, Candida, or Monilia genus, such as T. rubrum, T. interdigitale, T. tonsurans, M. audouinii, T. violaceum, M. ferrugineum, T. schoenleinii, T. megninii, T. soudanense, T. yaoundei, M. canis, T. equinum, T. erinacei, T. verrucosum, M. nanum, M. distortum, M. gypseum, and M. fulvum, which can infect the skin of a mammal and cause local swelling and inflammation thereof.

Accordingly, many topical anti-microbial formulations have previously been developed in an attempt to decrease or eliminate microbes, in particular dermatophytes, from the skin of an infected mammal, and to decrease or eliminate infection, inflammation, irritation, and/or discoloration associated with such dermatophyte infections.

Several of these previous topical anti-microbial formulations comprised an anti-microbial agent in a liquid composition with a pH above 8. However, it has been discovered that typical skin surfaces, even those infected with a dermatophyte infection, have a physiological pH in the range of about 4 to about 7.5. This physiological pH of the skin has been shown to play an important role in, e.g., skin rehabilitation, wound healing, reducing irritation, reducing infection processes, and determining the progression of dermatological fungal infections. See Rippke, F., “The acidic milieu of the horny layer: new findings on the physiology and patophysiology of skin pH”, Am. J. Clin. Dermatology, 2002,3(4): 261-72; Hachem JP, “pH directly regulates epidermal permeability barrier homeostasis, and stratum corneum integrity/cohesion”, J. Invest. Dermatol., 2003, 121(2): 45-53; and Runeman B., “Experimental Candida albicans lesions in healthy humans: dependence of skin pH”, Acta. Derm. Venereol., 2000, 80(6): 421-4.

Accordingly, several different anti-microbial compositions containing a lower pH have been proposed. For example, U.S. Pat. No. 6,231,875 to Sun et al. discloses methods for topical treatment of human nail and skin diseases, including fungal infections, bacterial infections, and psoriatic infections. In particular, Sun et al. disclose an acidified composition to treat nail and skin diseases in which the composition comprises at least one active agent, at least one acidifier, and at least one volatile solvent.

Similarly, U.S. Pat. No. 5,665,776 to Yu et al. discloses a composition for enhancing therapeutic effects of topically applied agents including one or more cosmetic or pharmaceutical agents present in a total amount of from 0.01 to 40 percent and one or more hydrocarboxylic acids in a total amount of from 0.01 to 99 percent by weight of the total composition. In particular, Yu et al. disclose that the cosmetic or pharmaceutical agents can be anti-bacterial, anti-fungal, anti-viral, and/or anti-dermatitis agents.

Additionally, U.S. Pat. No. 6,482,402 to Kurtz et al. discloses anti-microbial compositions for preventing or treating a microbial infection in a mammal containing a polymer and an anti-microbial agent. The polymer can be a homopolymer or a copolymer, and the anti-microbial agent can be an anti-bacterial agent, an anti-fungal agent, or an anti-septic.

However, the compositions disclosed by these patents tend to be harsh upon topical application to the skin of mammals. Anti-microbial agents in these previous topical compositions, and in general, have been known to dry, irritate, chafe, and/or inflame both the direct area being treated and the surrounding area. This is especially true with mammals having sensitive skin. This added negative stress to the skin can, and usually does, prolong the treatment and recovery time, and in some cases can worsen the microbial skin condition being treated to such an extent that the treatment has to be discontinued.

Skin protectants can alleviate some of these negative symptoms and side effects caused by the topical application of an anti-microbial agent to the skin of a mammal. Further, certain skin protectants can at times also aid in alleviating the negative symptoms caused by the disease being treated itself. In this regard, certain skin protectants can soften, smooth, and lubricate an infected and surrounding skin area by increasing the amount of water retained intercellularly with the layers of the skin, and by reducing the amount of transepidermal water loss (TEWL) experienced by the skin layers. Accordingly, skin protectants can help reduce treatment and recovery time, and even leave the previously infected skin area and surrounding areas in better condition than before the infection.

Certain skin protectants, especially low viscosity silicone compounds, however, are relatively unstable due to their volatile and/or flammable properties. Additionally, such skin protectants can be highly hydrophobic. Accordingly, it is often quite difficult to incorporate such low viscosity silicone compounds into anti-microbial compositions. This is especially true when the anti-microbial composition comprises a relatively voluminous aqueous phase. In this regard, skin protectants in general, especially low viscosity silicone compounds, can be relatively unstable, especially in emulsions or dispersion.

Additionally, many of the previous anti-microbial compositions were hindered by the low water solubility of the anti-microbial agent. Many anti-microbial agents are soluble under alkaline conditions, but promptly precipitate out of solution in crystalline form in acidic or slightly acidic conditions. As such, this often leads to problems relating to process control, as well as the overall stability and efficiency of the anti-microbial compositions. Accordingly, previously developed anti-microbial compositions usually either have 1) a neutral or basic pH to keep the anti-microbial agent from precipitating out of solution, or 2) a slightly acidic pH but a limited shelf life and efficiency due to the anti-microbial agent being unstable in an acidic solution. This limited shelf life and efficiency leads to increased economic costs for producing and continually replacing and restocking decomposed and possibly harmful anti-microbial compositions.

Moreover, the preparation of several anti-microbial compositions containing a skin protectant and an anti-microbial agent has been attempted. However, these compositions were often unable to incorporate a therapeutically effective amount of a pharmaceutically active anti-microbial agent with a relatively large amount of a skin protectant into a single, stable composition for topical application without diminishing or inactivating the advantageous properties of the skin protectant and/or the pharmaceutically active anti-microbial agent.

For these reasons, there remains a need in the art for stable topical anti-microbial compositions that are effective in temporarily or permanently reducing, inhibiting, treating, curing, or preventing a skin infection caused by microbes. Additionally, there remains a need in the art for such compositions that are also capable of reducing, inhibiting, treating, curing, or preventing discomfort, itching, and inflammation associated with microbial infections. Even more so, there remains a need in the art for compositions that provide stable topical anti-microbial compositions having a pH conducive to aiding in skin rehabilitation, wound healing, reducing irritation, and reducing infection processes. The present subject matter addresses these needs.

### SUMMARY OF THE INVENTION

The present subject matter relates generally to anti-microbial compositions, and more particularly to topical anti-microbial compositions.

In this regard, a preferred embodiment of the present subject matter relates to a topical anti-microbial composition comprising:

(i) a therapeutically effective amount of an anti-microbial agent;

(ii) a starch;

(iii) a hydroalcoholic solvent;

(iv) and a volatile silicone compound having a viscosity of about 0.1 to about 50 centistokes.

Another preferred embodiment of the present subject matter relates to a method of applying a therapeutically effective composition to a mammal which comprises:

a) preparing a lotion composition which comprises:
1) a therapeutically effective amount of an anti-microbial agent;
2) a starch;
3) a hydroalcoholic solvent; and
4) a volatile silicone compound having a viscosity of about 0.1 to about 50 centistokes;

b) applying the composition to an affected area on the mammal; and

c) allowing the solvent to evaporate from the composition to form a dry, powdery therapeutic composition.

A further preferred embodiment of the present subject matter relates to a method for preventing, inhibiting, or prophylactically treating a dermatophyte infection in a patient, which comprises administering to patient in need thereof a topical composition in an amount and manner sufficient to prevent, inhibit, or prophylactically treat said infection, said composition comprising:

(i) a therapeutically effective amount of an anti-microbial agent;

(ii) a starch;

(iii) a hydroalcoholic solvent; and

(iv) a volatile silicone compound having a viscosity of about 0.1 to about 50 centistokes.

Still another preferred embodiment of the present subject matter relates to a process for preparing a composition suitable for topical administration, said process comprising:
1) separately preparing an aqueous solvent composition and a hydroalcoholic solvent composition;
2) dissolving a therapeutically effective amount of an anti-microbial agent in said hydroalcoholic solvent composition;
3) adding said hydroalcoholic solvent composition to said aqueous solvent composition to obtain a mixture;
4) adding a starch to said mixture with mixing;
5) separately adding a volatile silicone compound having a viscosity of about 0.1 to about 50 centistokes to said mixture with mixing; and
6) recovering a composition suitable for topical administration.

### DETAILED DESCRIPTION OF THE INVENTION

### Definitions

As used herein, the terms “administering”, “administration”, and like terms refer to any method which, in sound medical or cosmetic practice, delivers a composition to a subject in such a manner as to provide a net positive effect on a dermatological disorder, condition, or appearance. The compositions are preferably administered such that they cover the entire area to be treated. “Direct administration” refers to any method which, in sound medical or cosmetic practice, delivers a composition to a subject without the use of another composition, delivery agent, or device. “Indirect administration” refers to any method which, in sound medical or cosmetic practice, delivers a composition to a subject with the use of at least one other composition, delivery agent, or device.

As used herein, the phrases an “effective amount” or a “therapeutically effective amount” of a pharmaceutically active anti-microbial agent or ingredient are synonymous and refer to an amount of the pharmaceutically active anti-microbial agent sufficient enough to have a positive effect on the area of application. Accordingly, these amounts are sufficient to modify the skin disorder, condition, or appearance to be treated but low enough to avoid serious side effects, within the scope of sound medical or dermatological advice. A therapeutically effective amount of the pharmaceutically active anti-microbial agent will cause a substantial relief of symptoms when applied repeatedly over time. Effective amounts of the pharmaceutically active anti-microbial agent will vary with the particular condition or conditions being treated, the severity of the condition, the duration of the treatment, the specific components of the composition being used, and like factors.

As used herein, the phrase “pharmaceutically acceptable salts” refers to salts of certain ingredient(s) which possess the same activity as the unmodified compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

If organic bases are used, poorly volatile bases are preferably employed, for example low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine. Ethanolamine is particularly preferred in this regard. Further poorly volatile bases which may be mentioned are, for example, ethylenediamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine.

Salts of quaternary ammonium hydroxides such as trimethylbenzylammonium hydroxide, tetramethylammonium hydroxide, or tetraethylammonium hydroxide can also by used, as can guanidine and its derivatives, in particular its alkylation products. However, it is also possible to employ as salt-forming agents, for example, low molecular weight alkylamines such as methylamine, ethylamine, or triethylamine. Suitable salts for the components to be employed according to the present subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained.

Other terms as used herein are meant to be defined by their well-known meanings in the art.

### Topical Anti-microbial Compositions

A preferred aspect of the subject matter expressed herein relates to various topical anti-microbial compositions. In this regard, the present subject matter preferably relates to a topical anti-microbial composition comprising:

(i) a therapeutically effective amount of an anti-microbial agent;

(ii) a starch

(iii) a hydroalcoholic solvent; and

(iv) a volatile silicone compound having a viscosity of about 0.1 to about 50 centistokes.

Such anti-microbial compositions are generally used to temporarily or permanently alleviate, reduce, inhibit, treat, ameliorate, or prevent anti-microbial infections such as seborrheic dermatitis, onychomycosis, ringworm of the foot, ringworm of the groin, sun fungus, and other external fungal infections.

### Anti-Microbial Agent

An essential component of the presently preferred compositions is a pharmaceutically active anti-microbial agent. The anti-microbial agent is capable of destroying or inhibiting the growth of microorganisms, and reducing, inhibiting, treating, ameliorating, or preventing discomfort, itching, and inflammation associated with microbial infections. The anti-microbial agent includes pharmaceutically active anti-bacterial agents, anti-fungal agents, anti-septic agents, anti-psoriatic agents, and derivatives and mixtures thereof. In particularly preferred embodiments, the anti-microbial agent is an anti-fungal agent or a derivative thereof.

In a preferred embodiment, the antimicrobial agents used in the present compositions possess anti-inflammatory properties. Accordingly, the present compositions possess anti-inflammatory properties as well. In particular, the antimicrobial agents, and thus the antimicrobial compositions, possess activity against microbes selected from the group consisting of gram positive bacteria, gram negative bacteria, funguses, molds, viruses, and combinations thereof.

The anti-microbial agent is preferably present in the instant compositions in a therapeutically effective amount. In this regard, the present compositions preferably contain about 0.01% to about 5% by weight, and more preferably from about 0.25% to about 3% by weight, of the anti-microbial agent.

Non-limiting examples of preferred anti-microbial agents useful herein include N-pyridine oxides, pharmaceutically acceptable salts thereof, and mixtures thereof. Particularly preferred N-pyridone oxides useful in this regard are those having the formula I: 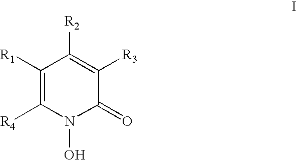
or a pharmaceutically acceptable salt thereof, wherein:

R₁, R₂, and R₃, which are identical or different, are H or alkyl having 1 to 4 carbon atoms, and R₄ is a saturated hydrocarbon radical having 6 to 9 carbon atoms or a radical of formula II: 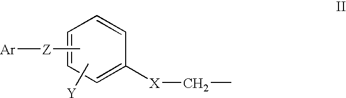
where:

X is S or O;

Y is selected from the group consisting of H, 1 or 2 identical halogen atoms, and a mixture of 2 different halogen atoms;

Z is selected from the group consisting of a single bond and a bivalent radical comprising O, S, CR₂ where R₂ is H or (C₁-C₄)-alkyl, or from 2 to 10 carbon atoms linked in the form of a chain, which optionally further comprises one or more of the following:
(i) a carbon-carbon double bond, or
(ii) O, S, or a mixture thereof, wherein if 2 or more O or S atoms or a mixture thereof are present, each O or S atom is separated by at least 2 carbon atoms; and,
in any of the foregoing bivalent radicals, free valences of the carbon atoms of said bivalent radical are saturated by H, (C₁-C₄)-alkyl, or a mixture thereof; and

Ar is an aromatic ring system having one or two rings that can be substituted by one, two, or three radicals, which may be identical or different, which are selected from the group consisting of halogen, methoxy, (C₁-C₄)-alkyl, trifluoromethyl, or trifluoromethoxy. These compounds are preferably present in the free or in the salt form.

In the radical “Z”, the carbon chain members are preferably CH₂ groups. If the CH₂ groups are substituted by C₁-C₄ alkyl groups, CH₃ and C₂H₅ are preferred substituents. Exemplary radicals “Z” are: —O—, —S—, —CH₂—, —(CH₂)ₘ— (m=2-10), —C(CH₃)₂—, —CH₂O—, —OCH₂—, —CH₂S—, —SCH₂—, —SCH(C₂H₅)—, —CH═CH—CH₂O—, —OCH₂CH═CHCH₂O—, —OCH₂CH₂O—, —OCH₂CH₂CH₂O—, —SCH₂CH₂CH₂S—, —SCH₂CH₂CH₂CH₂O—, —SCH₂CH₂OCH₂CH₂O—, —SCH₂CH₂OCH₂CH₂OCH₂CH₂S—, and —SCH₂C(CH₃)₂CH₂S—.

In the formula I, the hydrocarbon radical R₄ is preferably an alkyl or cyclohexyl radical which can also be bonded to the pyridone ring via a methylene or ethylene group or can contain an endomethyl group. R₄ can also be an aromatic radical which, however, is preferably bonded to the pyridone radical via at least one aliphatic carbon atom.

Preferred, non-limiting examples of the antimicrobial agent of formula I useful in the present compositions are those selected from the group consisting of: 6-[4-(4-chlorophenoxy)-phenoxymethyl]-1-hydroxy-4-methyl-2-pyridone, 6-[4-(2,4-dichlorophenoxy)phenoxymethyl]-1-hydroxy-4-methyl-2-pyridone, 6-(biphenyl-4-oxymethyl)-1-hydroxy-4-methyl-2-pyridone, 6-(4-benzyl-phenoxymethyl)-1-hydroxy-4-methyl-2-pyridone, 6-[4-(4-chlorophenoxy)phenoxymethyl]-1-hydroxy-3,4-dimethyl-2-pyridone, 6-[4-(2,4-dichlorobenzyl)phenoxymethyl]-1-hydroxy-4-3,4-dimethyl-2-pyridone, 6-[4-cinnamyloxyphenoxy methyl]-1-hydroxy-4-methyl-2-pyridone, 1-hydroxy-4-methyl-6-[4-(4-trifluoromethylphenoxy) phenoxymethyl]-2-pyridone, 1-hydroxy-4-methyl-6-cyclohexyl-2-pyridone, 1-hydroxy-4-methyl-6-(2,4,4-trimethylpentyl)-2-pyridone, 1-hydroxy-4-methyl-6-n-hexyl-, -6-iso-hexyl-, -6-n-heptyl-, or -6-isoheptyl-2-pyridone, 1-hydroxy-4-methyl-6-octyl- or -6-isooctyl-2-pyridone, 1-hydroxy-4-methyl-6-cyclohexylmethyl- or -6-cyclohexylethyl-2-pyridone, where the cyclohexyl radical can in each case also carry a methyl radical, 1-hydroxy-4-methyl-6-(2-bicyclo-[2,2,1]heptyl)-2-pyridone, 1-hydroxy-3,4-dimethyl-6-benzyl- or -6-dimethylbenzyl-2-pyridone, 1-hydroxy-4-methyl-6-(β-phenylethyl)-2-pyridone, a pharmaceutically acceptable salt thereof, and a mixture thereof.

In a particularly preferred embodiment, the antimicrobial agent of formula I useful in the present compositions is selected from the group consisting of: 6-[4-(4-chlorophenoxy)-phenoxymethyl]-1-hydroxy-4-methyl-2-pyridone, 1-hydroxy-4-methyl-6-cyclohexyl-2-pyridone, 1-hydroxy-4-methyl-6-(2,4,4-trimethylpentyl)-2-pyridone, a pharmaceutically acceptable salt thereof, and a mixture thereof.

In a most preferred embodiment, the antimicrobial agent of formula I used in the present compositions is 1-hydroxy-4-methyl-6-cyclohexyl-2-pyridone (Ciclopirox) or a pharmaceutically acceptable salt thereof. The ciclopiroxolamine salt is particularly preferred in this regard.

Antimicrobial agents other than those falling within formula I above are additionally contemplated as useful in the present antimicrobial compositions. Included among these other antimicrobial agents are those selected from the group consisting of imidazoles, allylamines, triazoles, glucan synthase inhibitors, chitin synthase inhibitors, polyenes, griseofulvin, morpholine derivatives, triazines, pyrimidines, any other antimicrobial azole, pharmaceutically acceptable salts thereof, and mixtures thereof. Other antimicrobial agents known in the art as effective upon topical administration to a patient are further contemplated as effective within the present compositions.

In a preferred embodiment, these other antimicrobial agents are those selected from the group consisting of amorolfine, amphotericin B, bacitracin, benzalkonium chloride, benzethonium chloride, bifonazole, butenafine, butoconazole, chloroxine, cilofungin, chlordantoin, chlortetracycline, clindamycin, clioqinol, clotrimazole, econazole, elubiol, faeriefungin, fezatione, fluconazole, flucytosine, fungimycin, gentamicin, griseofulvin, haloprogin, hexylresorcinol, itraconazole, ketoconazole, methylbenzethonium chloride, miconazole, mupirocin, naftifine, nikkomycin Z, nystatin, l-ofloxacin, oxiconazole, oxytetracycline, phenol, polymyxin B, pyrido[3,4-e]-1,2,4-triazine, pyrroInitrin, quaternary ammonium compounds, salicylic acid, saperconazole, sulconazole, tea tree oil, terbinafine, terconazole, tetracyclines, thiabendazole, ticlatone, tioconazole, tolnaftate, triacetin, triclocarbon, triclosan, undecylenic acid, voriconazole, zinc and sodium pyrithione, a pharmaceutically acceptable salt thereof, and a mixture thereof. Combinations of any of the foregoing anti-microbial agents or their pharmaceutically acceptable salts are contemplated herein.

### Starch

In addition to the pharmaceutically active anti-microbial agent, preferred embodiments of the present compositions further comprise a starch. The starch can enhance the effectiveness of the present compositions in temporarily or permanently reducing, inhibiting, treating, ameliorating, or preventing a skin infection caused by microbes. Additionally, the starch can provide a protective covering once the present compositions have been applied to the skin of a mammal, and can also serve as an emollient. Accordingly, the present compositions preferably contain about 1% to about 20% by weight of the starch. In a particularly preferred embodiment, the present compositions contain about 5 to about 15% by weight of the starch.

Non-limiting examples of preferred starches useful herein include maize starch, potato starch, rice starch, tapioca starch, wheat starch, corn starch, derivatives thereof, and mixtures thereof. Moreover, the starch can be pregelatinized or sterilizable. In a particularly preferred embodiment, the starch is tapioca starch.

### Hydroalcoholic Solvent

Further preferred embodiments of the present compositions comprise a hydroalcoholic solvent as an essential component. This hydroalcoholic solvent is preferably present in an amount from about 5% to about 20% by weight. In a more preferred embodiment, the present compositions comprise about 8% to about 14% by weight of the hydroalcoholic solvent.

The hydroalcoholic solvent is preferably a C₁-C₈, and more preferably a C₁-C₆ primary, secondary, or tertiary alcohol, or mixtures thereof. Non-limiting example of preferred hydroalcoholic solvents useful in this regard include methanol, ethanol, 1-propanol, 2-propanol, 1-butanol, 2-butanol, 1-pentanol, 2-pentanol, 3-pentanol, 1-hexanol, 2-hexanol, 3-hexanol, 1-heptanol, 2-heptanol, 3-heptanol, 4-heptanol, 1-octanol, 2-octanol, 3-octanol, 4-octanol, benzyl alcohol, derivatives thereof, and mixtures thereof.

### Volatile Silicone Compound

The present compositions further comprise a low viscosity volatile silicone compound. The volatile silicone compound can operate as a skin protectant in the present compositions. In this regard, the presently preferred anti-microbial compositions overcome many of the difficulties experienced by previous anti-microbial compositions containing a skin protectant in that they maintain stability over time despite the presence of a relatively high amount of the volatile silicone compound.

The volatile silicone compound used in the present compositions has a relatively low viscosity. In preferred embodiments, the volatile silicone compound used in the present compositions has a viscosity of about 0.1 to about 50 centistokes. In a particularly preferred embodiment, the volatile silicone compound has a viscosity of about 10 to about 40 centistokes.

The presently preferred compositions are unique in that they are formed as emulsions containing both an aqueous and oily phase, without diminishing the advantageous properties of the volatile silicone compound or the anti-microbial agent incorporated therein. Accordingly, the present preferred compositions overcome the difficulties often observed due to the hydrophobicity of many skin protectants. This permits the formation of compositions having a voluminous aqueous phase, which was previously difficult to accomplish without diminishing or inactivating the advantageous properties of the skin protectant and/or the pharmaceutically active anti-microbial agent. Accordingly, the present preferred compositions are advantageous over previous compositions that either do not contain, or contain lesser amounts of, a skin protectant, or are less stable and diminish the effectiveness of the skin protectant and the anti-microbial agent contained therein.

The volatile silicone compound, once applied to skin, can lower the transepidermal water loss (TEWL), or migration of moisture, through the deeper dermal tissues of the skin. Accordingly, by lubricating the skin, the volatile silicone compound of the present preferred compositions can lower the amount of TEWL experienced. This can alleviate and prevent negative symptoms caused by the skin infection disease or disorder being treated, as well as the negative side effects caused by the pharmaceutically active anti-microbial agent.

Accordingly, the presently preferred compositions can contain one or more volatile silicone compounds. In a preferred embodiment, the volatile silicone compound is a polysiloxane. In a more preferred embodiment, the polysiloxane is selected from the group consisting of (polydimethylsiloxane)ₙ and mixtures thereof, wherein n is from about 1 to about 10.

Preferred, non-limiting examples of polysiloxanes useful in this regard include polyorganosiloxanes, dimethicone, cyclomethicone, polydimethylsiloxanes, polydialkylsiloxanes, polydiarylsiloxanes, polyalkarylsiloxanes, octamethylcyclotetrasiloxane, decamethylcyclopentasiloxane, dodecamethyl-cyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, phenylsilicones, phenyltrimethicones, phenyldimethicones, phenyl-trimethylsiloxydiphenylsiloxanes, diphenyldimethicones, diphenylmethyldiphenyltrisiloxanes, 2-phenylethyltrimethyl siloxysilicates, derivatives thereof, and mixtures thereof. In a particularly preferred embodiment, the volatile silicone compound is dimethicone, such as Dow Fluid, 20CTS sold by Dow Corning Corporation.

In an alternative preferred embodiment, the volatile silicone compound can be an organosilicone such as a polyalkylsilicone, a cyclic polyalkylsiloxane, a polydialkylsiloxane, a polydiarylsiloxane, a polyalkarylsiloxane, or a cyclomethicone having 3 to 9 silicon atoms, and can be volatile or nonvolatile. Preferred polyalkylsiloxanes in this regard correspond to the general chemical formula R₃SiO[R₂SiO]SiR₃ wherein R₂ and R₃ are alkyl groups, while x is from about 0 to about 10. Additional examples of polyalkylsiloxanes useful herein include the Vicasil® series sold by General Electric Company and the Dow Corning® 200 series sold by Dow Corning Corporation.

Further preferred cyclic polyalkylsiloxanes useful in the present compositions include those corresponding to the general chemical formula [SiR₂O]ₙ wherein R₂ is an alkyl group and n is from about 1 to about 20, more preferably wherein n is from about 1 to about 10, and most preferably wherein n is from about 1 to about 8. When R₂ is methyl, these materials are typically referred to as cyclomethicones. Preferred, non-limiting examples of such cyclomethicones include Dow Corning® 244 fluid, which primarily contains the cyclomethicone tetramer (i.e. n=4), Dow Corning® 344 fluid, which primarily contains the cyclomethicone pentamer (i.e. n=5), Dow Corning® 245 fluid, which primarily contains a mixture of the cyclomethicone tetramer and pentamer (i.e. n=4 and 5), Dow Corning® 345 fluid, which primarily contains a mixture of the cyclomethicone tetramer, pentamer, and hexamer (i.e. n=4, 5, and 6), all sold by Dow Corning Corporation, derivatives thereof, and mixtures thereof.

In yet another alternative embodiment, the volatile silicone compound is a trimethylsiloxysilicate, which is a polymeric material corresponding to the general chemical formula [(CH₂)₃]ₓ[SiO₂]_{y}, wherein x is from about 1 to about 100 and y is from about 1 to about 100. An especially useful trimethylsiloxysilicate in this regard is Dow Corning® 593 fluid.

In a still further alternative embodiment, the volatile silicone compound can be a dimethiconol. Dimethiconols are hydroxy terminated dimethyl silicones, represented by the general chemical formulas R₅SiO[R₄SiO]ₓSiR₄OH and HOR₄SiO[R₄SiO]ₓSiR₄OH, wherein R₄ and R₅ are an alkyl group (preferably R₄ is methyl or ethyl, more preferably methyl) and x is an integer from 0 to about 500. Preferred, non-limiting examples of dimethiconols in this regard include mixtures with dimethicone or cyclomethicone, such as, but not limited to, Dow Corning® 1401, 1402, and 1403 fluids sold by Dow Corning Corporation.

In another alternative embodiment, the volatile silicone compound is a polyalkylaryl siloxane, which includes polymethyphenyl siloxane, such as SF 1075 methylphenyl fluid sold by General Electric Company and 556 Cosmetic Grade phenyl trimethicone fluid sold by Dow Corning Corporation.

### Additional Skin Protectant

In addition to the volatile silicone compound, the present compositions may optionally contain an additional skin protectant. Non-limiting examples of such additional skin protectants useful in this regard include petrolatum, red petrolatum, white petrolatum, liquid petrolatum, semi-solid petrolatum, light mineral oil, heavy mineral oil, white mineral oil, mineral oil alcohols, straight and branched chain hydrocarbons, C₁₀-C₃₀ alcohol esters of C₁₀-C₃₀ carboxylic acids, C₁₀-C₃₀ alcohol esters of C₁₀-C₃₀ dicarboxylic acids, monoglycerides of C₁₀-C₃₀ carboxylic acids, diglycerides of C₁₀-C₃₀ carboxylic acids, triglycerides of C₁₀-C₃₀ carboxylic acids, ethylene glycol monoesters of C₁₀-C₃₀ carboxylic acids, ethylene glycol diesters of C₁₀-C₃₀ carboxylic acids, propylene glycol monoesters of C₁₀-C₃₀ carboxylic acids, propylene glycol diesters of C₁₀-C₃₀ carboxylic acids, C₁₀-C₃₀ carboxylic acid monesters and polyesters of sugars, vegetable oils, hydrogenated vegetable oils, olive oil, hydrogenated olive oil, shea butter, polypropylene glycols, polypropylene glycol C₄-C₂₀ alkyl ethers, di C₈-C₃₀ alkyl ethers, synthetic hydrocarbons, derivatives thereof, and mixtures thereof.

In a preferred embodiment, the additional skin protectant is a straight or branched chain hydrocarbon having from about 7 to about 40 carbon atoms. Preferred, non-limiting examples of these hydrocarbon materials include dodecane, isododecane, squalane, cholesterol, hydrogenated polyisobutylene, docosane (i.e. a C₂₂ hydrocarbon), hexadecane, isohexadecane, derivatives thereof, and mixtures thereof. Also useful in this regard are the C₇-C₄₀ isoparaffins, which are C₇-C₄₀ branched hydrocarbons.

Preferred carboxylic acids useful as additional skin protectants in this regard include C₁₀-C₃₀ straight chain, branched chain, and aryl carboxylic acids, as well as propoxylated and ethoxylated drivatives of these carboxylic acids.

Preferred, non-limiting examples of vegetable oils and hydrogenated vegetable oils useful as additional skin protectants in this regard include safflower oil, caster oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, hydrogenated safflower oil, hydrogenated caster oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, olea europaea oil, hydrogenated olea europaea oil, palm glycerides, hydrogenated palm glycerides, derivatives thereof, and mixtures thereof.

Preferred, non-limiting examples of such additional skin protectants include diisopropyl sebacate, diisopropyl adipate, isopropyl myristate, isopropyl palmitate, myristyl propionate, ethylene glycol distearate, 2-ethylhexyl palmitate, isodecyl neopentanoate, C₁₂₋₁₅ alcohols benzoate, di-2-ethylhexyl maleate, cetyl palmitate, myristyl myristate, stearyl stearate, cetyl stearate, behenyl behenrate, dioctyl maleate, dioctyl sebacate, diisopropyl adipate, cetyl octanoate, diisopropyl dilinoleate, caprilic/capric triglyceride, PEG-6 caprylic/capric triglyceride, PEG-8 caprylic/capric triglyceride, PPG-14 butyl ether, PPG-15 stearyl ether, PPG-9, PPG-12, PPG-15, PPG-17, PPG-20, PPG-26, PPG-30, PPG-34, dioctyl ether, dodecyl octyl ether, derivatives thereof, and mixtures thereof.

Combinations of any of the foregoing skin protectants or their derivatives are further contemplated as useful in the present compositions.

### Aqueous Solvent

The presently preferred compositions can additionally comprise an aqueous solvent. In a preferred embodiment, the present compositions comprise about 25% to about 75% by weight of the aqueous solvent. In a more preferred embodiment, the present compositions comprise about 35% to about 65% by weight of the aqueous solvent. In a most preferred embodiment, the present compositions comprise about 40% to about 60% by weight of the aqueous solvent.

### Dermatologically Acceptable Excipients

The preferred compositions discussed herein can additionally comprise at least one dermatologically acceptable excipient commonly known to those of ordinary skill in the art as useful in topical compositions. Preferred, non-limiting examples of dermatologically acceptable excipients useful in these compositions are those selected from the group consisting of moisturizers/emollients, preservatives, gelling agents, colorants or pigments, anti-oxidants, radical scavengers, surfactants, emulsifiers, humectants, pH modifiers, chelating agents, derivatives thereof, and mixtures thereof.

### Moisturizers/Emollients

The presently preferred compositions may optionally further contain at least one moisturizer and/or emollient. Preferred non-limiting examples of moisturizers or emollients useful in this regard include glycerin, pentylene glycol, butylene glycol, polyethylene glycol, sodium pyrrolidone carboxylate, alpha-hydroxy acids, beta-hydroxy acids, polyhydric alcohols, ethoxylated and propoxylated polyols, polyols, polysaccharides, panthenol, hexylene glycol, propylene glycol, octyldodecanol, dipropylene glycol, sorbitol, derivatives thereof, and mixtures thereof. In a particularly preferred embodiment in this regard, the present compositions contain the emollient octyldodecanol.

When present, the emollient/moisturizer is preferably present in the instant compositions in about 5% to about 20% by weight.

### Preservatives

The presently preferred compositions may optionally further contain at least one preservative. Preferred non-limiting examples of preservatives useful in this regard include propylene glycol, glycerol, butylene glycol, pentylene glycol, hexylene glycol, sorbitol, benzyl alcohol, ethanol, derivatives thereof, and mixtures thereof.

The preservative is preferably present in an amount of about 0.1% to about 2.5% by weight of the overall weight of the composition.

### Gelling Agents

The presently preferred compositions may optionally further contain at least one gelling agent. Preferred, non-limiting examples of gelling agents useful in this regard include various cellulose agents, such as hydroxyethylcellulose, cellulose gum, methylcellulose, hydroxypropylcellulose, hydroxypropylmethylcellulose, xanthan gum, gum arabic, gum tragacanth, locust bean gum, guar gum, other cellulosic polymers, derivatives thereof, and mixtures thereof.

Other suitable gelling agents useful in the present compositions include sodium carbomer, carbomer, polyacrylic polymers, aqueous gelling agents, such as neutral, anionic, and cationic polymers, derivatives thereof, and mixtures thereof.

Exemplary polymers which may be useful in the preferred compositions include carboxy vinyl polymers, such as carboxypolymethylene. In this regard, a preferred gelling agent is a Carbopol® polymer (i.e. a polyacrylic polymer) such as is available from Noveon Inc., Cleveland, Ohio. Another particularly preferred gelling agent is a polyacrylic polymer, for example a copolymer of acrylic acid and a long chain alkyl methacrylate. This copolymer can be crosslinked with polyalkenyl ethers of polyalcohols, for example as with a Pemulen® polymer available from Noveon Inc., Cleveland, Ohio. In this regard, the polyacrylic polymer or any other gelling agent is preferably present in the instant compositions in an amount of from about 0.01% to about 10%, more preferably from about 0.05% to about 5%, and most preferably from about 0.1% to about 2%, by weight.

### Anti-Oxidants

The presently preferred compositions may optionally further contain at least one anti-oxidant. Preferred non-limiting examples of antioxidants useful in this regard include ascorbic acid, ascorbyl esters of fatty acids, magnesium ascorbyl phosphate, sodium ascorbyl phosphate, ascorbyl sorbate, tocopherol, tocopherol sorbate, tocopherol acetate, butylated hydroxy benzoic acid, thioglycolates, persulfate salts, 6-hydroxy-2,5,7,8-tetramethylchroman-2-carboxylic acid, lipoic acid, gallic acid, propyl gallate, uric acid, sorbic acid, lipoic acid, amines, N,N-diethylhydroxylamine, N-acetyl-L-cysteine, amino-guanidine, sulfhydryl compounds, glutathione, dihydroxy fumaric acid, lycine pidolate, arginine pilolate, nordihydroguaiaretic acid, bioflavonoids, curcumin, lysine, 1-methionine, proline, superoxide dismutase, silymarin, tea extracts, grape skin/seed extracts, melanin, rosemary extracts, derivatives thereof, and mixtures thereof.

### Surfactants

The presently preferred compositions may optionally further contain at least one surfactant. Preferred non-limiting examples of surfactants useful in this regard include zwitterionic surfactants, amphoteric surfactants, anionic surfactants, cationic surfactants, nonionic surfactants, and mixtures thereof. Preferred zwitterionic, amphoteric, anionic, cationic, and nonionic surfactants include those disclosed in McCutcheon's, Detergents and Emulsifiers, North American edition (1986), published by allured Publishing Corporation, and McCutcheon's, Functional Materials, North American Edition (1992), both of which are incorporated by reference herein in their entirety.

### Emulsifiers

The presently preferred compositions may optionally further contain an emulsifier. Preferred non-limiting examples of emuslifiers useful in this regard include any of a wide variety of nonionic, cationic, anionic, zwitterionic and amphoteric emulsifiers.

Preferred, non-limiting examples of specific emulsifiers useful in this regard include glycol esters, fatty acids, fatty alcohols, fatty acid glycol esters, fatty esters, fatty ethers, esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, stearic acid, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, derivatives thereof, and mixtures thereof.

In a preferred embodiment, the present compositions can comprise about 0.01% to about 15% by weight of an emulsifier. In a more preferred embodiment, the present compositions can comprise about 0.1% to about 10% by weight of an emulsifier.

### Humectants

The presently preferred compositions may optionally further contain a humectant. Preferred, non-limiting examples of humectants useful in this regard include sorbitol, sorbitol syrup, E965 maltitol, maltitol, maltitol syrup, E1200 polydextrose, E1518 glyceryl triacetate, triacetin, glyceryl triacetate, 1,2,3-propanetriyl triacetate, 1,2,3-propanetriol triacetate, triacetylglycerol, E1520 propylene glycol, 1,2-propanediol, 1,2-dihydroxypropane, methylethylene glycol, propane-1,2-diol, E420 sorbitol, propylene glycol, polyethylene glycol (PEG) esters, PEG-20 stearate, PEG-40 stearate, PEG-150 stearate, PEG-150 distearate, PEG-100 stearate, laureth-12, ceteareth-20, laureth-23, glycereth-7, glycereth-12, glycereth-26, PEG-4, PEG-6, PEG-8, PEG-12, PEG-32, PEG-75, PEG-150, derivatives thereof, and mixtures thereof.

In a preferred embodiment, the present compositions can comprise about 0.1% to about 10% by weight of a humectant. In a more preferred embodiment, the present compositions can comprise about 0.5% to about 5% by weight of a humectant.

### pH Modifiers

The presently preferred compositions may optionally further contain a pH modifier. Preferred non-limiting examples of pH modifiers useful in this regard include inorganic hydroxides, inorganic oxides, inorganic salts of weak acids, inorganic acids, organic acids, derivatives thereof, and mixtures thereof.

Preferred, non-limiting examples of inorganic hydroxides useful in this regard include ammonium hydroxide, alkali metal hydroxide, alkaline earth metal hydroxides, derivatives thereof, and mixtures thereof.

Preferred inorganic hydroxides useful herein include ammonium hydroxide, monovalent alkali metal hydroxides such as sodium hydroxide and potassium hydroxide, divalent alkali earth metal hydroxides such as calcium hydroxide and magnesium hydroxide, derivatives thereof, and mixtures thereof.

Preferred, non-limiting examples of inorganic oxides useful herein include magnesium oxide, calcium oxide, derivatives thereof, and mixtures thereof.

Preferred, non-limiting examples of inorganic salts of weak acids useful herein include ammonium phosphate (dibasic), alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic), alkaline earth metal salts of weak acids such as magnesium phosphate and calcium phosphate, derivatives thereof, and mixtures thereof.

Preferred, non-limiting examples of inorganic acids useful herein include hydrochloric acid, hydrofluoric acid, hydrobromic acid, nitric acid, nitrous acid, hydrocyanic acid, perchloric acid, chlorous acid, sulfurous acid, hypochlorous acid, phosphoric acid, acetic acid, sulfuric acid, derivatives thereof, and mixtures thereof.

Preferred, non-limiting examples of organic acids useful herein include lactic acid, citric acid, glutamic acid, methanoic acid, ethanoic acid, phenol, monochloroethanoic acid, dichloroethanoic acid, trichloroethanoic acid, butanoic acid, salicylic acid, glycolic acid, and mixtures thereof.

Further, mixtures of any of the above-mentioned pH modifiers are also contemplated as within the scope of the present compositions.

### Chelating Agents

The presently preferred compositions may optionally further contain a chelating agent. Preferred non-limiting examples of chelating agents useful in this regard include citric acid, isopropyl (mono) citrate, stearyl citrate, lecithin citrate, gluconic acid, tartaric acid, oxalic acid, phosphoric acid, sodium tetrapyrophosphate, potassium monophosphate, sodium hexametaphosphate, calcium hexametaphosphate, sorbitol, glycine (aminoacetic acid), methyl glucamine, triethanolamine (trolamine), EDTA, DEG (dihydroxyethylglycine), DPTA (diethylene triamine pentaacetic acid), NTA (Nitrilotriacetic Acid), HEDTA (N-(hydroxyethyl)-ethylenetriaminetriacetic acid), aminocarboxylates, dimercaperol (BAL), larixinic acid (Maltol), unidentate ligands (fluoride and cyanide ions), diphenylthiocarbazone, 0-phenanthroline, barium diphenylamine sulfonate, sodium glucoheptonate, 8-hydroxyquinoline, olefin complexes (such as dicyclopentadienyl iron), porphyrins, phosponates, pharmaceutically acceptable salts thereof, derivatives thereof, and mixtures thereof.

In addition to those enumerated above, any other anti-microbial agent, starch, hydroalcoholic solvent, volatile silicone compound, additional skin protectant, moisturizer, emollient, preservative, gelling agent, colorant or pigment, antioxidant, radical scavenger, surfactant, emulsifier, humectant, pH modifier, chelating agent, or other dermatologically acceptable excipient commonly known to those of ordinary skill in the art as useful in topical compositions is contemplated as useful in the compositions described herein. Further, any non-toxic, inert, and effective topical carrier may be used to formulate the compositions described herein.

Well-known carriers used to formulate other topical therapeutic compositions for administration to humans will be useful in these compositions. Examples of these components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, http://www.accessdata.fda.gov/scripts/cder/iig/index.cfm, the contents of which are hereby incorporated by reference in their entirety. Examples of such useful pharmaceutically acceptable excipients, carriers and diluents include distilled water, physiological saline, Ringer's solution, dextrose solution, Hank's solution, and DMSO, which are among those preferred for use herein.

These additional other inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

In another particularly preferred embodiment, the presently preferred pharmaceutical compositions are formulated in a lotion, cream, ointment, gel, suspension, emulsion, foam, aerosol, or other pharmaceutically acceptable topical dosage form. A lotion is particularly preferred in this regard.

### Stabibility and Pharmacological Activity

The presently preferred topical pharmaceutical compositions are further unique in that they are storage stable with respect to the anti-microbial agent, as well as the other components present in the compositions. Accordingly, these compositions have a decided advantage over previous anti-microbial compositions in that they limit the amount of degradation experienced by each component of the composition over time, resulting in a composition with improved long-term efficacy at temperatures of about 30° C. or below. In this regard, the present compositions are preferably able to maintain a purity of at least 90% and a concentration of degradation product(s) less than about 10% of the starting concentration of the anti-microbial agent.

Further, the remarkable stability of the preferred compositions solves long felt difficulties in formulating compositions having an anti-microbial agent and a skin protectant, such as a volatile silicone compound. Since these compositions have an increased stability over the previous known compositions in the art, they provide unexpected advantages over the prior art compositions. For example, the increased storage stability permits the presently preferred compositions to be manufactured in greater quantities without fear that the compositions produced will be wasted. Further, the enhanced stability provides the presently preferred compositions with an enhanced effect in reducing, inhibiting, treating, ameliorating, or preventing microbial infections treatable with an anti-microbial agent over the previously known compositions.

The selection of specific excipients and amounts thereof in the presently preferred compositions, as well as the preparation of compositions having a specific designated pH in the form of a designated emulsion, conveys these unique stability characteristics to the presently preferred compositions.

In addition to the unique stability characteristics of the presently preferred anti-microbial compositions, these compositions further have a unique pH, which allows skin to rehabilitate and heal quicker while reducing irritation, inhibiting the infection process, and having a positive effect on the progression of dermatological fungal infections. In this regard, the current preferred compositions have a pH in the range of about 4 to about 7.5, while maintaining about 90%, and preferably about 95%, of the anti-microbial agent in its soluble, pharmaceutically active form. This allows the present compositions to efficiently deliver a therapeutically effective amount of an anti-microbial agent to the skin of a mammal, while maintaining an optimum pH level to reduce, inhibit, treat, ameliorate, or prevent discomfort, itching, inflammation and/or discoloration associated with the microbial infection. In this regard, the present compositions preferably have a pH of about 6.5 to about 7.5.

The pH of skin plays an important role in skin rehabilitation, wound healing, reducing irritation, reducing infection processes, and the progression of dermatological microbial infections. Accordingly, by providing the anti-microbial agent in a high, unprecipitated, non-degraded form at a more natural skin physiological pH then previous compositions, the current compositions can reduce, inhibit, treat, ameliorate, or prevent a microbial infection in a two fold manner. In this regard, the pharmaceutically active anti-microbial agent can 1) more efficiently reduce, inhibit, treat, ameliorate, or prevent a microbial infection while 2) the skin is kept at a natural physiological pH level to aid in efficiently reducing, inhibiting, treating, ameliorating, or preventing a microbial infection.

### Methods of Treatment

Another preferred aspect of the present subject matter pertains to a method of applying a therapeutically effective composition to a patient, which comprises

a) preparing a lotion composition which comprises:
(i) a therapeutically effective amount of an anti-microbial agent;
(ii) a starch;
(iii) a hydroalcoholic solvent; and
(iv) a volatile silicone compound having a viscosity of about 0.1 to about 50 centistokes;

b) applying said composition to an affected area on the mammal; and

c) allowing the solvent to evaporate from the composition to form a dry, powdery therapeutic composition.

In this regard, the preferred compositions described herein can be used in methods for temporarily or permanently reducing, inhibiting, treating, ameliorating, or prophylactically treating skin microbial infections, as well as other skin diseases or disorders, and restoring or repairing a skin lipid barrier of a mammal. These methods can be achieved by topically applying the presently preferred compositions to the skin of a patient, such as a mammal. In this regard, the present compositions can be used in methods for preventing, inhibiting, or prophylactically treating a dermatophyte infection.

The present compositions are effective when applied to a patient either directly or indirectly. In a preferred embodiment, the present compositions are directly applied to the area of skin to be treated. In an alternative preferred embodiment, the present compositions are indirectly applied to the area of skin to be treated. Such indirect application can occur via, for example, an article of clothing, a pad, or a pledget. In this regard, the article of clothing can be absorbent or non-absorbent.

In preferred embodiments, the administration of the present compositions reduces the number of microbes, preferably pathogenic microbes, on the skin of the mammal to which it is applied. The microbes that can be acted on by the present antimicrobial shampoos are selected from the group consisting of bacteria, funguses, molds, viruses, and combinations thereof.

Preferred examples of bacteria treatable with the present compositions are gram positive bacteria, gram negative bacteria, and combinations thereof. Specific, non-limiting examples of such gram positive bacteria are those selected from the group consisting of Streptococcus sp., Micrococcus sp., Staphylococcus sp., Bacillus sp., and combinations thereof.

Preferred, non-limiting examples of such Streptococcus sp. are those selected from the group consisting of S. viridans, S. agalactiae, S. pyogenes, S. faecalis, S. durans, S. faecium, S. mutans, S. sanguis, S. salivarius, S. mitior, S. constellatus, S. intermedius, S. anginosus, S. milleri, S. iniae, S. pneumoniae, and combinations thereof.

Preferred, non-limiting examples of such Staphylococcus sp. are those selected from the group consisting of S. aureus, S. epidermidis, and combinations thereof.

Preferred, non-limiting examples of such fungi are those selected from the group consisting of P. ovale, P. oviculare, M. furfur, T. beigelii, B. capitatus, P. marneffei, C. neoformans, S. prolificans, S. shenkii, Epidermophyton floccosum, Microsporum canis, Candida sp., Trichophyton sp., and combinations thereof.

Preferred, non-limiting examples of such Candida sp. are those selected from the group consisting of C. albicans, C. cruzii, C. krusei, C, glabrata, C. guillermondii, C. inconspicua, C. parapsilosis, C. tropicalis, and combinations thereof.

Preferred, non-limiting examples of such Trichophyton sp. are those selected from the group consisting of T. rubrum, T. mentagrophytes, T. tonsurans, T. violaceum, and combinations thereof.

Preferred, non-limiting examples of such molds are those selected from the group consisting of Aspergillus sp., B. dermatitidis, P. brasiliensis, and combinations thereof.

Preferred, non-limiting examples of such Aspergillus sp. are those selected from the group consisting of A. flavus, A. fumigates, A. niger, and combinations thereof.

Several specific skin disorders may also be treated according to the present inventive methods. Exemplary among these skin disorders are seborrheic dermatitis, Pityrosporum infections, tinea versicolor, tinea pedis, tinea cruris, tinea corporis, cutaneous candidiasis, and combinations thereof. Other skin disorders known to those of ordinary skill in the art as effectively treatable by a topical composition are further contemplated as within the scope of the present subject matter.

Further, the present inventive methods provide antimycotic activity against Pityrosporum strains, such as Pityrosporum ovale and Pityriasis versicolor.

In this regard, the present compositions are particularly effective in treating the specific dermatophytes Tinea and/or Candida fungi.

Further, the volatile silicone compound of the present compositions can function as an emollient, lubricating the skin and lowering the amount of TEWL experienced by layers of the skin. All of these functions allow the present compositions to temporarily or permanently alleviate skin microbial infections and/or other skin disorders experienced by the mammal. Additionally, the anti-microbial agent alleviates itchy, painful, irritated sensations caused by the microbial infections, such as topical fungal and bacterial infections.

The presently preferred compositions can further fortify the skin lipid barrier to prevent its disruption due to a microbial infection and/or environmental insults. In this regard, once topically applied to the skin of a mammal, the preferred compositions can lubricate the skin, increase intercellular adhesion in the skin of the mammal, and lower the TEWL experienced by the skin. This increased intercellular adhesion can result in the restoration and/or repair of the skin lipid barrier, which can aid in reducing, inhibiting, treating, ameliorating or preventing microbial infections and other skin disorders.

This repair of the skin lipid barrier can improve the skin barrier function which increases the skin's ability to aid in reducing, inhibiting, treating, ameliorating or preventing microbial infections, and can convey numerous additional therapeutic effects to a mammal to which the preferred compositions are applied. In addition to reducing, inhibiting, treating, ameliorating or preventing microbial infections, this skin lipid barrier repair can further enhance the repair of the skin to which the compositions are applied, increase the interstitial oil content of the skin, improve the integrity of the skin's interstitial lipid layer, treat additional skin disorders such as additional microbial infections, and reduce the occurrence of further skin barrier malfunctions. The increased interstitial oil content of the skin and the improved integrity of the skin's interstitial lipid layer can be a direct result from the enhanced skin repair. Accordingly, the present anti-microbial compositions are unexpectedly useful in methods of treating mammalian microbial infection skin areas, and surrounding areas.

In addition to and concurrently with the skin repair, the increased intercellular adhesion resulting from administration of the present compositions further reduces manifestations of microbial infections while enhancing the skin repair. This reduction of microbial manifestations is optimally achieved by daily topically applying the preferred compositions to the skin of a mammal. These compositions are superior to those compositions presently available for the reduction of microbial infections, and thus for the normalization and moisturization of the skin, due to their extended therapeutic characteristics. Accordingly, the presently preferred compositions can provide both an immediate therapeutic effect, as well as an extended therapeutic effect.

Moreover, the present compositions provide a topical composition, which upon application to a surface of skin, allows a starch film to be deposited on the affected skin and surrounding area to help alleviate, reduce, inhibit, treat, ameliorate, or prevent a microbial infection and the symptoms and side effects of the same. This starch film additionally protects the infected and surrounding area by acting as an intermediate layer between these areas and environmental stresses and/or clothing. The starch film is initially deposited by the evaporation and/or absorption of the hydroalcoholic solvent and absorption of the skin protectant into the infected and surrounding area of the skin. Accordingly, the present compositions provide a therapeutically effective amount of an anti-microbial agent and a skin protectant, along with a starch component, which forms a delayed protective starch film to cover the affected and surround areas of a microbial infection.

### Combination Therapy

In another preferred embodiment, the present preferred compositions may be used in combination with an additional pharmaceutical dosage form to enhance their effectiveness in treating a dermatological disease or disorder. In this regard, the present preferred compositions may be administered as part of a regimen additionally including any other pharmaceutical and/or pharmaceutical dosage form known in the art as effective for the treatment of a dermatological disorder. Similarly, a pharmaceutically active anti-microbial agent other than those specified herein can be added to the present preferred compositions to enhance their effectiveness in treating a dermatological disease or disorder. Accordingly, this additional anti-microbial agent or additional pharmaceutical dosage form can be applied to a patient either directly or indirectly, and concomitantly or sequentially, with the preferred compositions described herein.

In one embodiment in this regard, the present preferred composition and the additional pharmaceutical dosage form can be administered to a patient at the same time. In an alternative embodiment, one of the present preferred compositions and the additional pharmaceutical dosage form can be administered in the morning and the other can be administered in the evening.

### Methods of Production

Another preferred aspect relates to a process for preparing a composition suitable for topical administration.

In this regard, the present preferred processes can be carried out in various steps. For example, one preferred process herein relates to a process for preparing a composition suitable for topical administration, said process comprising:
1) separately preparing an aqueous solvent composition and a hydroalcoholic solvent composition;
2) dissolving a therapeutically effective amount of an anti-microbial agent in said hydroalcoholic solvent composition;
3) adding said hydroalcoholic solvent composition to said aqueous solvent composition to obtain a mixture;
4) adding a starch to said mixture with mixing;
5) separately adding a volatile silicone compound having a viscosity of about 0.1 to about 50 centistokes to said mixture with mixing; and
6) recovering a composition suitable for topical administration.

In a preferred embodiment in this regard, the aqueous solvent composition is prepared by mixing one or more gelling agents in an aqueous solvent. In particularly preferred embodiments, the one or more gelling agents can be a cellulose agent, such as xanthan gum, a polymer, such as a copolymer of acrylic acid and a long chain alkyl methacrylate cross-linked with polyalkenyl ethers of polyalcohols, or a mixture thereof.

In another preferred embodiment, the hydroalcoholic solvent composition is prepared by first mixing a moisturizer/emollient in a hydroalcoholic solvent before dissolving a therapeutically effective amount of the anti-microbial agent therein. In a particularly preferred embodiment, the hydroalcoholic solvent used in this regard is selected from the group consisting of a C₁-C₆ alcohol, benzyl alcohol, and mixtures thereof.

In a further preferred embodiment, the hydroalcoholic solvent composition is added to the aqueous solvent composition while mixing to obtain the mixture. In another preferred embodiment, a pH modifier is added to said mixture prior to the separate additions of said starch and said volatile silicone compound.

It would be expected that these processes herein described will permit the anti-microbial agent to remain in solution throughout the process and in the final topical composition.

The present processes preferably form compositions comprising an emulsion having an oil. phase and an aqueous phase. Non-limiting examples of specific types of emulsions that can be made according to this process include an oil-in-water emulsion, a water-in-oil emulsion, an oil-in-water-in-oil emulsion, and a water-in-oil-in-water emulsion. The formation of a specific type of emulsion will depend on the specific ingredients used in the process. In a preferred embodiment, the process will form compositions that are oil-in-water emulsions.

This particular preparation process is a non-limiting example of a possible process that can be used to prepare the preferred compositions. Other processes capable of preparing these compositions are further contemplated herein. Further, the individual phases of the preferred compositions (for example aqueous and oil phases) can be prepared sequentially in any order or concurrently; it is not necessary to prepare the oil phase before the aqueous phase is prepared in order to practice the present processes. Additionally, preferred compositions can be prepared according to either a batch process or continuously.

Further contemplated as within the scope of the present subject matter are pharmaceutical compositions produced according to the above-described process. If produced according to this process, these compositions exhibit chemical and physical stability suitable for topical administration.

The compositions produced according to these processes can be placed in a suitable containment vessel comprising a product contact surface composed of a material selected from the group consisting of glass, plastic, Teflon, polymeric structure, and mixtures thereof. These containment vessels are used to facilitate manufacturing, handling, processing, packaging, storage, and administration of said composition. Preferred containment vessels in this regard can be selected from the group consisting of plastic tubes and bottles.

### Dosage

Appropriate dosage levels for the pharmaceutically active anti-microbial agents contemplated in the preferred compositions and methods are well known to those of ordinary skill in the art and are selected to maximize the treatment of microbial skin infections and the above-mentioned skin conditions. Dosage levels on the order of about 0.001 mg to about 5,000 mg per kilogram body weight of the pharmaceutically active anti-microbial agent and skin protectant components are known to be useful in the treatment of the diseases, disorders, and conditions contemplated herein. Typically, this effective amount of the pharmaceutically active anti-microbial agent and skin protectant components will generally comprise from about 0.001 mg to about 100 mg per kilogram of patient body weight per day. Moreover, it will be understood that this dosage of ingredients can be administered in a single or multiple dosage units to provide the desired therapeutic effect.

If desired, other therapeutic agents can be employed in conjunction with those provided in the above-described compositions. The amount of anti-microbial ingredients that may be combined with the carrier materials to produce a single dosage form will vary depending upon the host treated, the nature of the disease, disorder, or condition, and the nature of the anti-microbial ingredients.

The preferred pharmaceutical compositions may be given in a single or multiple doses daily. In a preferred embodiment, the pharmaceutical compositions are given from one to three times daily. The amount of anti-microbial ingredients that may be combined with the carrier materials to produce a single dosage form will vary depending upon the host treated, the nature of the disease, disorder, or condition, and the nature of the anti-microbial ingredients.

It is understood, however, that a specific dose level for any particular patient will vary depending upon a variety of factors, including the activity of the specific pharmaceutically active anti-microbial ingredient and skin protectant; the age, body weight, general health, sex and diet of the patient; the time of administration; the rate of excretion; possible drug combinations; the severity of the particular condition being treated; and the form of administration. One of ordinary skill in the art would appreciate the variability of such factors and would be able to establish specific dose levels using no more than routine experimentation.

The optimal pharmaceutical formulations will be determined by one skilled in the art depending upon considerations such as the particular skin protective ingredient and pharmaceutically active agent combination and the desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the essential lipids.

### EXAMPLES

The following examples are illustrative of preferred compositions and are not intended to be limitations thereon. All polymer molecular weights are mean average molecular weights. All percentages are based on the percent by weight of the final delivery system or formulation prepared unless otherwise indicated and all totals equal 100% by weight.

### Example 1

The following example illustrates the preparation of a present preferred lotion:

| | % W/W |
|---|---|
| Purified Water | 48.57 |
| Carbomer Co-polymer | 0.3 |
| Xanthan Gum | 0.2 |
| Ciclopirox Olamine, USP | 1.948 |
| Octyldodecanol | 12.0 |
| Benzyl Alcohol | 1.5 |
| Dehydrated Alcohol (Ethanol) | 10.0 |
| Sodium Hydroxide | 0.01 |
| Lactic Acid | 0.47 |
| Dimethicone | 15.0 |
| Starch Tapioca | 10.0 |
| | 100.0% |

Preparation of the Lotion:

An aqueous solvent composition is prepared by mixing the purified water, the xanthan gum, and the carbomer copolymer.

A hydroalcoholic solvent composition is prepared by first mixing the benzyl alcohol, dehydrated alcohol, and octyldodecanol in a separate vessel. The Ciclopirox Olamine is then added to this vessel and dissolved in the hydroalcoholic solvent mixture.

The hydroalcoholic solvent composition is then added to the aqueous solvent composition with mixing. The sodium hydroxide and lactic acid are then added to this mixture with mixing, followed by the starch tapioca and dimethicone, one at a time, with mixing.

### Example 2

The following example illustrates the preparation of a present preferred lotion:

| | % W/W |
|---|---|
| Purified Water | 49.12 |
| Carbomer Co-polymer | 0.3 |
| Xanthan Gum | 0.2 |
| Ciclopirox Olamine, USP | 1.299 |
| Octyldodecanol | 12.0 |
| Benzyl Alcohol | 1.5 |
| Dehydrated Alcohol (Ethanol) | 10.0 |
| Sodium Hydroxide | 0.11 |
| Lactic Acid | 0.47 |
| Dimethicone | 15.0 |
| Starch Tapioca | 10.0 |
| | 100.0% |

This lotion is prepared according to the process described above for Example 1.

### Example 3

A patient is suffering from a microbial skin infection. A preferred composition herein is topically administered to the patient. It would be expected that the patient would improve his/her condition or recover.

### Example 4

A patient is suffering from a Tinea infection. A preferred composition herein is topically administered to the patient. It would be expected that the patient would improve his/her condition or recover.

### Example 5

A patient is suffering from a Candida infection. A preferred composition herein is topically administered to the patient. It would be expected that the patient would improve his/her condition or recover.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

What is claimed is:

1. A topical lotion composition comprising:
(i) a therapeutically effective amount of an anti-microbial agent;
(ii) a starch;
(iii) a hydroalcoholic solvent; and
(iv) a volatile silicone compound having a viscosity of about 0.1 to about 50 centistokes.

2. The composition of claim 1, wherein said anti-microbial agent is an anti-fungal agent or a derivative thereof.

3. The composition. of claim 2, wherein said anti-fungal agent is selected from the group consisting of N-pyridine oxides, pharmaceutically acceptable salts thereof, and mixtures thereof.

4. The composition of claim 3, wherein said anti-fungal agent is selected from the group consisting of ciclopirox, ciclopirox olamine, derivatives thereof, and mixtures thereof.

5. The composition of claim 1, comprising about 0.1% to about 5.0% by weight of said anti-microbial agent.

6. The composition of claim 1, wherein said starch is selected from the group consisting of maize starch, potato starch, rice starch, tapioca starch, wheat starch, corn starch, derivatives thereof, and mixtures thereof.

7. The composition of claim 6, wherein said starch is tapioca starch.

8. The composition of claim 1, comprising about 1% to about 20% by weight of said starch.

9. The composition of claim 1, wherein said hydroalcoholic solvent is selected from the group consisting of a C₁-C₆ alcohol, benzyl alcohol, and mixtures thereof.

10. The composition of claim 9, wherein said C₁-C₆ alcohol is selected from the group consisting of a primary alcohol, a secondary alcohol, a tertiary alcohol, and mixtures thereof.

11. The composition of claim 9, wherein said hydroalcoholic solvent is selected from the group consisting of ethanol, propanol, butanol, pentanol, benzyl alcohol, and mixtures thereof.

12. The composition of claim 1, comprising about 5% to about 20% by weight of said hydroalcoholic solvent.

13. The composition of claim 1, wherein said volatile silicone is a polysiloxane.

14. The composition of claim 13, wherein said polysiloxane is selected from the group consisting of (polydimethylsiloxane)ₙ and mixtures thereof, wherein n is from 1 to 10.

15. The composition of claim 1, further comprising a copolymer of acrylic acid and a long chain alkyl methacrylate cross-linked with polyalkenyl ethers of polyalcohols.

16. The composition of claim 14, comprising about 0.1% to about 2% of said copolymer of acrylic acid and a long chain alkyl methacrylate cross-linked with polyalkenyl ethers of polyalcohols.

17. The composition of claim 1, further comprising an emollient.

18. The composition of claim 17, comprising about 5% to about 20% of said emollient.

19. A method of applying a therapeutically effective composition to a mammal which comprises:
a) preparing a lotion composition which comprises:
(i) a therapeutically effective amount of an anti-microbial agent;
(ii) a starch;
(iii) a hydroalcoholic solvent; and
(iv) a volatile silicone compound having a viscosity of about 0.1 to about 50 centistokes;
b) applying said composition to an affected area on the mammal; and
c) allowing the solvent to evaporate from the composition to form a dry, powdery therapeutic composition.

20. A method for preventing, inhibiting, or prophylactically treating a dermatophyte infection in a patient, which comprises:
administering the composition of claim 1 to a patient in need thereof in an amount and manner sufficient to prevent, inhibit, or prophylactically treat said infection.

21. The method of claim 20, wherein said administering is done directly or indirectly.

22. The method of claim 20, wherein said dermatophyte is selected from the group consisting of a Trichophyton fungi, a Candida fungi, a Tinea fungi, M. furfur, and combinations thereof.

23. A process for preparing a composition suitable for topical administration, said process comprising:
1) separately preparing an aqueous solvent composition and a hydroalcoholic solvent composition;
2) dissolving a therapeutically effective amount of an anti-microbial agent in said hydroalcoholic solvent composition;
3) adding said hydroalcoholic solvent composition to said aqueous solvent composition to obtain a mixture;
4) adding a starch to said mixture with mixing;
5) separately adding a volatile silicone compound having a viscosity of about 0.1 to about 50 centistokes to said mixture with mixing; and
6) recovering a composition suitable for topical administration.

24. The process of claim 23, wherein the aqueous solvent composition is prepared by mixing one or more gelling agents in an aqueous solvent.

25. The process of claim 23, wherein the hydroalcoholic solvent composition is prepared by first mixing an emollient in a hydroalcoholic solvent before said dissolving of said therapeutically effective amount of the anti-microbial agent.

26. The process of claim 23, wherein a pH modifier is added to said mixture prior to the separate additions of said starch and said volatile silicone compound.

27. The process of claim 23, wherein the anti-microbial agent remains in solution throughout the process and in the final topical composition.
